(19) United States
(12) Patent Application Publication
(10) Pub. No.: US 2026/0083371 A1
(43) Pub. Date: Mar. 26, 2026

(54) Portable Neck Assessment System and Methods

(71) Applicant: University of North Carolina at Wilmington, Wilmington, NC (US)

(72) Inventors: Alexander T. McDaniel, Wilmington, NC (US); Lindsey H. Schroeder, Wilmington, NC (US)

(73) Assignee: University of North Carolina at Wilmington, Wilmington, NC (US)

(21) Appl. No.: 19/400,980

(22) Filed: Nov. 25, 2025

Related U.S. Application Data
(63) Continuation-in-part of application No. 17/511,216, filed on Oct. 26, 2021, now Pat. No. 12,478,306.
(60) Provisional application No. 63/105,623, filed on Oct. 26, 2020.

(51) Int. Cl. A61B 5/22; A61B 5/00; A63B 21/002

(52) U.S. Cl. CPC A61B 5/224; A61B 5/0022; A61B 5/4566; A61B 5/6814; A61B 5/6831; A61B 5/702; A61B 5/7267; A61B 5/7275; A63B 21/0023; A61B 2562/0219; A61B 2562/0252

(57) ABSTRACT

A portable neck strength assessment system and methods associated therewith are provided. A portable neck strength assessment system includes an isometric and dynamic neck strength device including a tension measurement component, a risk assessment module configured to generate an assessment of an injury risk of an individual, the assessment of the injury risk based at least in part on a minimum threshold determined by an artificial intelligence model or a machine learning model, an anchor line having a first end and a second end, a tension line having a first end and a second end, and a wearable head harness, where the second end of the anchor line is in operable communication with the isometric neck strength device, and the first end of the tension line is in operable communication with the isometric neck strength device, and a second end of the tension line is connected to the wearable head harness.

### RELATED APPLICATION DATA

This application is a continuation-in-part of U.S. application Ser. No. 17/511,216, titled “PORTABLE ISOMETRIC NECK ASSESSMENT SYSTEM AND METHOD,” filed Oct. 26, 2021, which claims priority under 35 U.S.C. § 119 (e) to U.S. Provisional Patent Application No. 63/105,623 filed Oct. 26, 2020, the contents of each are incorporated by reference herein in their entireties.

### FIELD

The technology described herein generally relates to systems, devices, and methods for assessing neck strength, and more specifically to portable neck strength measurement and assessment systems, devices, and methods.

### BACKGROUND

Mild traumatic brain injury (mTBI) is a common injury which calls for accurate diagnosis as it may have clinical consequences that can become more problematic for a patient without proper interventions and therapies. Evaluation and rehabilitation for mTBI's can, in part, rely on neck strength assessment protocols.

TBI's can result from multiple situations, including falls, motor vehicle crashes, sports injuries, and assaults. Recently, there has been increasing interest in exploring the association between neck strength and the risks of mTBI's to develop strength-based prevention protocols. Evidence suggests that increased neck strength can lead to decreased head acceleration, decreased head displacement, and decreased rapid velocity changes following a collision, which when taken together may decrease the risk of severity of mTBI's. It has further been seen that those who sustain mTBI show a decrease in overall neck strength.

Currently, conventional fixed-frame dynamometry provides a reliable method and “standard” for assessing neck strength, however, it may be impractical for use in many clinical settings due to its high-expense and non-transportable nature. For example, fixed-frame dynamometry devices have multiple limitations as they are generally large wall or frame mounted machines with a fixed base. Further, known protocols that use portable hand-held systems lack standardization and reliable implementation, and as such, while portable they do not produce reliable and consistent data. Additionally, conventional neck assessment products lack features which enable robust portability and compatibility or usage with other clinical devices and applications.

Accordingly, the technology described herein can overcome issues in conventional devices, systems, and methods of neck and/or neck strength assessments and enhance the quality of care delivered to patients and enable clinicians to deliver more robust assessments, rehabilitation, and risk mitigation procedures through portable and inexpensive devices and systems.

### SUMMARY

This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

Embodiments of the technology described herein are directed towards reliable and portable neck strength assessment systems, devices, and methods that can achieve a high confidence interval with respect to neck strength assessments, and additionally be used in conjunction with one or more health risk assessment protocols.

According to some aspects, a portable neck strength assessment system is provided. An example portable neck strength assessment system can include an isometric neck strength device including a tension measurement component. The portable neck strength assessment system further includes a risk assessment module configured to generate an assessment of an injury risk of an individual using an artificial intelligence model or a machine learning model. The portable neck strength assessment system further includes an anchor line having a first end and a second end. The portable neck strength assessment system further includes a tension line having a first end and a second end. The portable neck strength assessment system further includes a wearable head harness, where the second end of the anchor line is in operable communication with the isometric neck strength device, and where the first end of the tension line is in operable communication with the isometric neck strength device, and a second end of the tension line is connected to the wearable head harness.

According to some aspects, a portable neck strength assessment system is provided. An example portable neck strength assessment system can include a neck strength measurement device including a tension measurement component. The portable neck strength assessment system further includes a risk assessment module configured to generate an assessment of an injury risk of an individual using an artificial intelligence model or a machine learning model. The portable neck strength assessment system further includes an anchor line having a first end and a second end. The portable neck strength assessment system further includes a tension line having a first end and a second end. The portable neck strength assessment system further includes a wearable head harness, where the second end of the anchor line is in operable communication with the isometric neck strength device, and where the first end of the tension line is in operable communication with the isometric neck strength device, and a second end of the tension line is connected to the wearable head harness. In some aspects, the system can determine and/or measure both isometric neck strength and dynamic neck strength, where in some instances isometric neck strength is based on a subject's static neck movement and dynamic neck strength is based on a subject's response to an anticipated and/or unanticipated neck pull on the harness system.

In some embodiments of the portable neck strength assessment system, the injury risk of the individual is further based at least in part on a neck strength determined using the isometric neck strength device.

In some embodiments of the portable neck strength assessment system, the tension measurement component comprises at least one transducer that measures neck strength.

In some embodiments of the portable neck strength assessment system, the tension measurement component includes at least one tension transducer that measures neck strength.

In some embodiments of the portable neck strength assessment system, the system further includes an inertia transducer that characterizes movement of the isometric neck strength device. In some embodiments of the portable neck strength assessment system, the inertia transducer is an accelerometer.

In some embodiments of the portable neck strength assessment system, the isometric neck strength device comprises a static strength measuring system and a dynamic strength measuring system.

In some embodiments of the portable neck strength assessment system, the artificial intelligence model or the machine learning model generates the assessment of the injury risk based on anticipated assessment data and unanticipated assessment data.

In some embodiments of the portable neck strength assessment system, the portable neck strength assessment system further includes, a linear actuator between two rings comprising a first ring at the first end and a second ring at the second end, and a transducer that adjusts a distance between the two rings.

In some embodiments of the portable neck strength assessment system, the portable neck strength assessment system further includes a microcontroller that measures at least one transducer signal of the isometric neck strength device.

In some embodiments of the portable neck strength assessment system, the portable neck strength assessment system further includes a mobile application executing on a mobile device, wherein the mobile application receives data from the isometric neck strength device and uploads the data to a remote storage.

In some embodiments of the portable neck strength assessment system, the portable neck strength assessment system further includes a web application accessed on a computing device, wherein the web application provides access to uploaded data captured via the isometric neck strength device to a database remote from the computing device.

In some embodiments of the portable neck strength assessment system, the portable neck strength assessment system further includes an application executing on a computing device, wherein the application provides at least one user interface comprising instructions for performing an isometric strength assessment using the isometric neck strength device and the wearable head harness.

In some embodiments of the portable neck strength assessment system, the portable neck strength assessment system further includes an application executing on a computing device, wherein the application provides at least one user interface comprising instructions for calibrating the portable neck strength assessment system for use. In some embodiments of the portable neck strength assessment system, the portable neck strength assessment system is calibrated using a set of standardized weights.

In some embodiments of the portable neck strength assessment system, the portable neck strength assessment system further includes an application executing on a computing device, wherein the application comprises an algorithm that uses isometric neck strength data captured using the isometric neck strength device to determine a resistance to use for dynamic neck strength assessment for an individual.

In some embodiments of the portable neck strength assessment system, the risk assessment module includes an application executing on a computing device, wherein the application comprises an algorithm that determines at least one risk factor based at least in part on a neck strength measured for an individual using the isometric neck strength device.

In accordance with a second aspect of the disclosure, a method for injury risk assessment is provided. An example method includes measuring a neck strength using an isometric neck strength device that performs an isometric neck strength assessment. The method further includes generating a minimum threshold using an artificial intelligence model or a machine learning model. The method further includes determining an assessment of an injury risk based on the minimum threshold and the neck strength.

In some embodiments of the method, the method further includes displaying the assessment of the injury risk via an application executed on a mobile computing device.

In some embodiments of the method, the method further includes displaying the neck strength via an application executed on a mobile computing device, wherein the isometric neck strength device communicates with the mobile computing device to provide the neck strength.

In some embodiments of the method, the method further includes transmitting, by the isometric neck strength device to the mobile computing device, the neck strength, and uploading, by the mobile computing device using an application executed on the mobile computing device, the neck strength to a database remote from the computing device.

In some embodiments of the method, the method further includes adjusting a distance of the isometric neck strength device to a wearable head harness using at least one transducer.

In some embodiments of the method, the machine learning model or artificial intelligence model is based at least in part on static strength assessment data and dynamic strength assessment data.

In some embodiments of the method, the machine learning model or the artificial intelligence model is based at least in part on anticipated assessment data and unanticipated assessment data.

Additional objects, advantages, and novel features of the invention will be set forth in part in the description which follows, and in part will become apparent to those skilled in the art upon examination of the following, or can be learned by practice of the technology.

### BRIEF DESCRIPTION OF THE DRAWINGS

Aspects of the technology described herein are described in detail below with reference to the accompanying drawing figures, which are not necessarily drawn to scale, wherein:

FIG. 1 illustrates a block diagram of an example portable neck strength assessment system in accordance with some aspects of the technology described herein;

FIG. 2 illustrates a schematic diagram of an example operating environment for an neck strength assessment system, in accordance with some implementations of the technology described herein;

FIG. 3 illustrates a schematic diagram of an example neck strength assessment system, in accordance with some implementations of the technology described herein;

FIG. 4 is a flow diagram showing a method for implementing a portable isometric assessment system, in accordance with some aspects of the technology described herein;

FIG. 5 is a block diagram of an example computing environment and/or device architecture in which some implementations of the present technology may be employed;

FIG. 6 is a block diagram of system components performing static and dynamic neck strength measuring in accordance with at least some aspects of the technology described herein; and

FIG. 7 is another block diagram of another example portable neck strength assessment system measuring in accordance with at least some aspects of the technology described herein.

### DETAILED DESCRIPTION

The subject matter of aspects of the present disclosure is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” can be used herein to connote different elements of methods employed, the terms should not be interpreted as implying any particular order among or between various steps disclosed herein unless and except when the order of individual steps is explicitly described.

Accordingly, embodiments described herein can be understood more readily by reference to the following detailed description, examples, and figures. Elements, apparatus, and methods described herein, however, are not limited to the specific embodiments presented in the detailed description, examples, and figures. It should be recognized that the exemplary embodiments herein are merely illustrative of the principles of the invention. Numerous modifications and adaptations will be readily apparent to those of skill in the art without departing from the spirit and scope of the invention.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7. All ranges disclosed herein are also to be considered to include the end points of the range, unless expressly stated otherwise. For example, a range of “between 5 and 10” or “5 to 10” or “5-10” should generally be considered to include the end points 5 and 10. Further, when the phrase “up to” is used in connection with an amount or quantity; it is to be understood that the amount is at least a detectable amount or quantity. For example, a material present in an amount “up to” a specified amount can be present from a detectable amount and up to and including the specified amount.

Aspects of the technology disclosed herein will now be described more fully with reference to the accompanying drawings, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated drawings. Therefore, it is to be understood that the presently disclosed subject matter is not to be limited to the specific embodiments disclosed and that modifications and other embodiments are intended to be included within the scope of the appended claims.

At a high level, aspects of the present technology generally relate to neck strength assessment and/or measurement systems, devices, and methods. According to some aspects, a neck strength assessment can include one or both of an isometric strength assessment (also referred to as a static strength assessment) and a dynamic strength assessment. In some aspects a Neck Strength Assessment Tool (NSAT)1, NSAT system, or neck strength assessment system is provided that can incorporate both hardware and/software to perform a neck strength assessment, which may include one or more of an isometric strength assessment and/or a dynamic strength assessment.

According to some aspects, a neck strength assessment tool or system can be a portable, non-invasive, assessment system that can gather both static and dynamic neck strength data and also accurately identify and individual (e.g. a patient, subject) at risk of sustaining a head/neck injury. In some aspects, systems and methods described herein predict a risk level for an individual or a probability of sustaining an injury. According to some aspects, a neck assessment system (or neck strength assessment system) incorporates one or more measurement sensors (e.g. on-board measurement sensors) and a device (or tool) interface which provides multiple advantages over existing assessment tools or systems, such as optimization for ease of us, portability, and efficient deployment in a given setting (e.g. clinical setting, at a user's home, in the field, etc.). In some further aspects, a neck assessment system (or neck strength assessment system) is run utilizing a control unit or controllers (which can be integrated into one or more devices or portions of the system) which interfaces with a digital application, such as a mobile application running on a user device (e.g. smart phone, tablet, etc.).

In some instances, a digital application can implement, utilize, and/or leverage Artificial Intelligence (AI) and/or Machine Learning (ML) algorithms or models to enable or enhance functionality, accuracy, and/or effectiveness of the system. In some instances, an algorithm or model for the system can leverage a neural network or interconnected nodes, which in some further aspects can be trained on one or more sets of training data, for example training data that encompasses traumatic brain injury (TBI) data or datasets.

As will be appreciated, the integration of AI or ML algorithms and/or models enable improved capture of critical neuromuscular data, for example more specifically, data related to static and/or dynamic force production and attenuation. Without intending to be bound by theory, such data can be vital for monitoring the health of the head/neck complex, especially in response to bodily impact or whiplash-induced injuries. By processing and analyzing this data, the system disclosed herein can identify individuals at high risk for head and neck trauma, thus enabling preventive measures and contributing to the enhancement of resilience and physical readiness of an individual.

In some aspects, systems and methods herein provide neck assessments (or neck strength assessments) which can incorporate isometric strength assessments and dynamic strength assessments. In some instances, these assessments are portable and can sync with an application (e.g. digital application). In some aspects, isometric strength assessments can provide a baseline of a neck strength assessment to an application and for a subsequent dynamic neck strength assessment. In some aspects, a dynamic neck strength assessment can determine time to contraction for one or both of anticipated and unanticipated neck pull applied to a subject. In other words, the actual neck strength will counteract a force experienced coming from a neck strength assessment device. Further, in some aspects, isometric and dynamic neck strength values can be used as inputs to an AI or machine learning algorithm or model that can predict a potential risk of head and/or neck trauma (e.g. TBI) based on one or more anthropomorphic measurements.

In some instances, an application or implemented software in the portable system can assess anthropomorphic measurements and/or metrics to detect or predict a potential risk of head and/or neck trauma, or for instance a TBI, and provide a user or subject of the measurement system a clear indication above or below a determined threshold for return to normal activity. As will be appreciated, systems and methods described herein provides an early detection mechanism that can prevent future injuries and improve overall safety of an individual or subject.

Systems and methods herein provide portable, reliable, and/or rapid neck strength assessments (i.e. provides a neck strength assessment tool) that can determine a risk of head/neck injury (e.g. TBI) based on static and dynamic neck strength measurements. In some aspects, a neck strength assessment system can comprise a controller, one or more sensors, a mobile application, a static strength measurement system, a dynamic strength measurement system, and a risk metric algorithm.

In some aspects, a controller can interface with the measurements systems (static measurement system, dynamic measurement system), the one or more sensors, and the mobile application. In some aspects, the controller can incorporate various hardware and software components for operation, such as power management and circuit boards. As further described herein, a static measurement system (or component) can interface with a patient or subject through a harness system (e.g. wearable harness, tension line(s), load measurement device) to determine a static neck strength, or one or more static neck strength measurements, of a patient in multiple directions. As further described herein, a dynamic measurement system (or component) can interface with a patient or subject through a harness system to determine a dynamic neck strength, or one or more dynamic neck strength measurements of a subject in multiple directions. According to some aspects, the dynamic measurement system (or component) uses the static measurements from the static measurement system (or component) to set the forces used in the dynamic strength measurement. The dynamic system or component can quantify dynamic neck strength and time to contraction through, for instance multiple assessments, including an anticipated pull and/or an unanticipated pull.

According to embodiments of the technology described herein, neck strength assessment systems, devices, and methods are provided, for example for use in conjunction with patient rehabilitation, mild traumatic brain injury (mTBI) prevention and risk mitigation, outcomes, and neck strengthening protocols. Neck strength systems and/or devices as described herein may be implemented, for instance, as assessment or pre-assessment tools in a rehabilitation setting and/or strengthening or performance enhancement setting, or prior to more invasive interventions. In some embodiments, isometric neck strength assessment devices can be mobile, easy to operate, for instance in providing assessments, and inexpensive. According to some aspects, neck strength assessment tools, systems, and/or protocols described herein can incorporate one or both of isometric (or static) assessments and/or dynamic assessments. As such, assessment systems or protocols described herein can incorporate an isometric component and/or a dynamic component. In some aspects both a static and a dynamic assessment are used or combined to provide an output, such as a risk level for an injury or a protocol to reduce the risk of an injury. In some aspects, a neck strength assessment system described herein is comprised of both static (isometric) and dynamic strength measuring systems. In some aspects these systems can be integrated into an application (i.e. digital application) with incorporated or built-in AI/ML algorithms for risk prediction.

Accordingly, systems, devices, and methods are provided for measuring or assessing neck strength, for instance by a portable neck strength assessment device. Systems and devices described herein are inexpensive and further have been demonstrated to be over 90% effective as conventional standard neck assessment tools in four planes of motion. As such, the technology described herein provides neck strength measurement devices and assessment systems that are integrated units, which are portable and incorporate wireless features and are mobile application capable, and maintain high levels of reliability and validity in providing a user with neck strength assessment results and metrics. In some aspects, neck strength assessment systems and/or devices (e.g. portable neck strength assessment systems) provide for the measurement of static neck strength through isometric measurements, and dynamic neck strength through anticipated and unanticipated forces.

According to some embodiments, a neck strength assessment device and/or system is provided, for example as an assessment tool to measure isometric neck strength. Isometric neck strength can be measured in the following movements by a user: cervical flexion, cervical extension, capital flexion, capital extension, left lateral flexion, right lateral flexion, left cervical rotation, and right cervical rotation. In some instances, isometric neck strength can be measured in one or more, or all of the forgoing movements, and in some other instances, isometric neck strength can be measured by a subset of the forgoing movements. For example, in some embodiments, isometric neck strength can be measured using a subset of capital flexion, cervical extension, left cervical lateral flexion, and right cervical lateral flexion. As will be appreciated, in some aspects, neck strength in any direction can be measured in any appropriate manner for force determination, such as Newtons (N) for force or Newton-meters (Nm) for torque which are useful in both applying force to the head and/or neck in various directions (e.g. flexion, extension, rotation) and taking and or recording the resulting strength measurement of a subject.

According to some further embodiments, a neck strength assessment device or system includes a neck strength measurement device, which in some instances can incorporate a strain gauge and/or tension scale which can measure and/or providing output readings of neck strength based on neck movements of a user. A neck strength measurement device can be connected to an anchor line, which can be affixed to a stationary object and can further be connected to a tension line, which can be affixed to a head harness worn by a user while performing a given neck movement. Based on a user performing a neck movement, a measure of neck strength may be taken, for instance by the isometric neck strength measurement device, and transmitted or otherwise communicated as data to a remote computing device or application running on a computing device. The neck strength measurement device may collect and transmit any number of other mobility measurements in some embodiments, for example other data associated representing directions, speeds, strengths, or other information associated with movements.

The neck strength may be characterized by the application, assessed with respect to a particular risk factor, facilitate research, and/or the like. The neck strength in some embodiments is communicated using one or more wireless communications protocol, for example Bluetooth Low Energy. In some embodiments, additionally or alternatively, the application running on the computing device uploads or otherwise forwards the data (e.g., neck strength measurement) to one or more dataset. In some embodiments, the database is a remote (or “cloud”) database, which is separate from the application and/or computing device.

Current research suggests that strengthening neck muscles can reduce the risk of sustaining acute head and neck injuries, including TBI. Studies primarily focused on linear acceleration forces, however, which may not fully account for multi-directional forces experienced by warfighters in dynamic environments, such as parachute landings, or by high-risk athletes in contact sports. The use in predicting TBI risk is thus limited. An alternative metric for accurate TBI risk assessment is thus needed, for example to account for rotational acceleration in impacts and/or other complex multi-directional force environments. To determine a more complete metric for TBI risk, a combination of patient linear static neck strength and rate of force development (RFD) can be used. Linear static strength is a measurement of neck strength taken in the various directions of head motion. RFD measures how quickly the neck can create the specific, coordinated force needed to stabilize the head (e.g., dynamic neck strength). An algorithm can then use the measured patient static and dynamic neck strength to determine a risk metric for the patient that can be used to conclude if the patient is at risk for a TBI. A risk metric in some embodiments is determined and utilized to generate a corresponding protocol to increase neck strength and/or to reduce the injury risk accordingly.

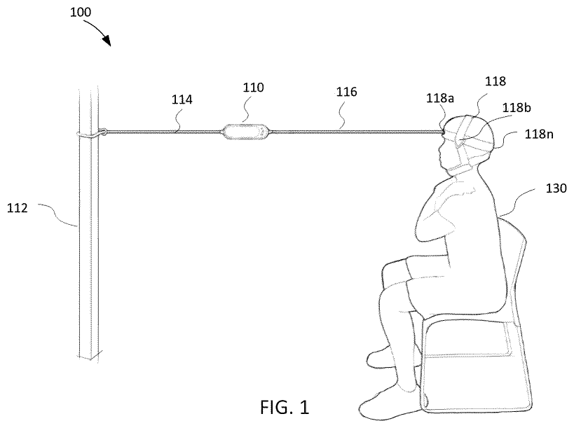

FIG. 1 depicts a block diagram of an example portable neck assessment system 100 (also referred to herein as a neck strength assessment system) in accordance with various embodiments of the present technology. Neck strength assessment system 100 can include an neck strength measurement device 110, an anchor line 114, a tension line 116 and a headgear apparatus 118. It will be appreciated that in some instances, neck strength assessment device may refer to a combination or sub-combination of any of these components in addition to other components described herein. The neck strength measurement device can incorporate a tension scale, strain gauge, one or more load cells, and/or another suitable transducer configured to convert, for example, energy, force, torque, and/or motion produced by a user into one or more electrical signals which can then be used by one or more control systems, for instance. In some aspects, the measurement device herein is referred to as an isometric neck strength measurement device which can be implemented to measure isometric neck strength of a user. The same device can similarly be implemented with embodiments of the present technology to provide dynamic neck strength measurements (e.g. correlated to user or subject reaction to anticipated or unanticipated neck pulls, which can be generated by the system or an individual administering one or more of the measurement tests).

One side of the neck strength measurement device 110 can be anchored to a stationary object 112 via an anchor line 114. Anchor line 114 can be made of any material suitable for use in neck assessment system 100, for instance for performing an neck assessment, such as a cable material, nylon based material, or a combination thereof. The other side of the neck strength measurement device 110 can be connected, anchored, or otherwise attached to a head harness/strap 118 worn by a user 130 via a tension line 116. It will be appreciated that with respect to FIG. 1, user 130 is the subject of the neck assessment or neck strength assessment. Anchor line 114 and tension line 116 can be integrated with and/or otherwise attached to isometric and/or dynamic neck assessment device 110 such that an isometric action by user 130 (e.g. muscle contraction) will cause neck strength measurement device 110 (also described herein a isometric and/or dynamic neck strength measurement device) to produce or otherwise generate a reading or measurement corresponding to one or more isometric and/or dynamic neck assessment tests and/or neck strength tests. In some embodiments, isometric and/or dynamic neck strength measurement device 110 is capable of measuring up to, for example, about 30 pounds of force. Further, isometric and/or dynamic neck strength measurement device 110 may include a plurality of mechanisms for connecting anchor line 114 and tension line 116, such as shackles, S-hooks, D-rings, or other suitable connection mechanisms. In some other embodiments, anchor line 114 and tension line 116 are integrated (e.g. directly integrated) with a housing body of isometric and/or dynamic neck strength measurement device 110, and are connected (either physically or operably) with a strain gauge/tension scale, or other transducer of the neck strength measurement device 110. Additionally, tension line 116 may be similarly attached to or otherwise integrated with headgear 118. Anchor line 114 can similarly be attached and/or otherwise affixed to a stationary object 112, or in some examples be formed into a loop and secured to the stationary object 112. Head harness/strap 118 can additionally comprise multiple connection points 118a, 118b, 118n which enable different isometric and/or dynamic neck strength measurements to be taken. For instance, tension line 116 can be connected to or otherwise attached to a front portion, side portion, or back portion of head harness strap 118. In some embodiments, custom body interfaces are provided that align the neck strength measurement device in at least one specific direction. Such directions may be utilized in assessing neck strengths with respect to various directions.

It will be appreciated that the positioning and/or configuration of the head harness/strap 118 on the user 130 and/or the user 130 with respect to the neck strength measurement device 110 may vary based on a specific neck assessment or strength test. For instance, in one example embodiment, a portable neck strength assessment system 100 can be configured such that user 130 is standing and isometric neck strength measurement device 110 is arranged off the side of the user 130's head harness/strap 118. In another example embodiment, portable neck strength assessment system 100 can be configured such that user 130 is sitting and isometric neck strength measurement device 110 is arranged off the front of the user 130's head harness/strap 118. In some embodiments, the system 100 is used with the user 130 standing while wearing the head harness/strap 118. Both standing and sitting provides sufficient reliability between measurements of a user. The user 130 may, in some embodiments, be positioned in any manner, for example including positions other than directly facing or facing away from the corresponding instrumentation to enhance consistency of the performance of the system 100 across users and uses.

In some embodiments, the portable neck strength assessment system 100 is portable in a manner that can be moved throughout evaluation areas. In some embodiments, the portable neck strength assessment system 100, or a portion thereof for example a controller, is connected to a power source (e.g., an AC outlet) upon being moved to the evaluation area. In some embodiments, one or more sensors of the portable neck strength assessment system 100 are configured to be connectable to the controller of the portable neck strength assessment system 100.

In some embodiments, the system 100 includes or interfaces with one or more systems that measure particular data points associated with the user 130. For example, in some embodiments, the isometric neck strength measurement device 110 includes or is associated with a static system that, through the harness, determines the static neck strength of the user 130 in one or more directions. Additionally or alternatively, in some embodiments, the isometric neck strength measurement device 110 includes or is associated with a dynamic system that, interfaces with the user 130 to determine a dynamic neck strength of the user 130 in multiple directions. In some embodiments, the dynamic system utilizes the static measurements from a static system to set one or more forces used in a dynamic strength measurement. For example, a pull level may be set for a particular direction, such that a pull force is applied by the dynamic strength system 608 until the sensor measuring the reaction force reaches the set pull level for that direction. In some embodiments, a dynamic system quantifies dynamic neck strength and time-to-contraction, for example using assessments such as an anticipated pull assessment and/or an unanticipated pull assessment.

The system described herein may be utilized by any of a myriad of user groups. For example, in some contexts, sports medicine professionals, such as athletic trainers, utilize the system for prevention, assessment, treatment, and/or rehabilitation of athletically-related injuries. In some contexts, rehabilitative specialists, such as physical therapists, physiotherapists, occupational therapists, and/or the like, may use the system for rehabilitation of any of a number of musculoskeletal ailments. In some contexts, physicians, physician assistants, or other medical professionals utilize the system in a multitude of areas including orthopedics and sports medicine. In some contexts, strength and conditioning coaches utilize the system to train athletes to improve athletic performance. In some contexts, exercise physiologists utilize the system to improve injury resistance. In some contexts, personal trainers utilize the system to help clients achieve desired health and fitness goals, for example by designing and implementing exercise programs that utilize the system. Users of these groups may be responsible for equipment set-up, assisting in implementation of protocols, managing the associated application (e.g., a mobile application), and/or monitoring performance. The system depicted and described herein may be used in any of such contexts and environments, and the portable nature of the system enables the system to be relocated and set up in any such environments.

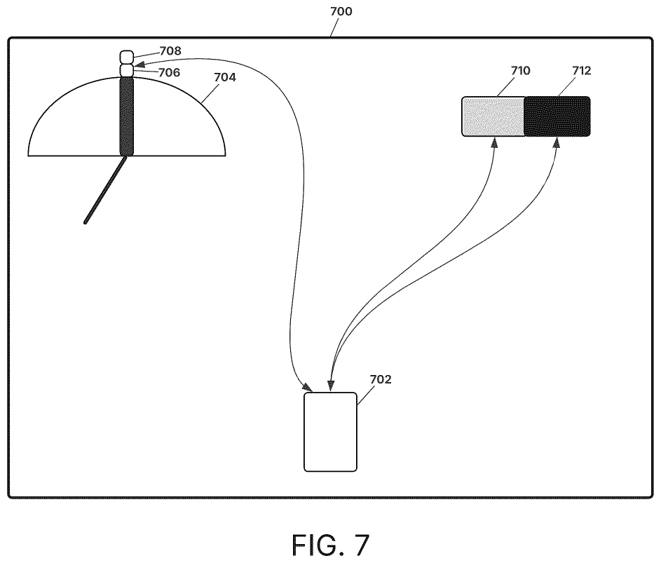

FIG. 7 depicts another block diagram of another example portable neck strength assessment system 700, in accordance with various embodiments of the present technology. Specifically, FIG. 7 depicts a system 700 including a controller 702. The controller 702 is communicatively coupled (e.g., connected via a wire, for example) with one or more components of the head harness 704. For example, in some embodiments, the head harness 704 includes or otherwise is connected to a 3-axis angular rate sensor 706 and a 3-axis accelerometer 708. The 3-axis angular rate sensor 706 is specially configured to measure and/or report measurements of rates of angular rotation measured during manipulation of the head harness 704, for example pitch, roll, and yaw values. In some embodiments, the 3-axis angular rate sensor 706 is an inertial sensor, gyroscope, and/or the like. The 3-axis accelerometer 708 in some embodiments measures acceleration along each axis. The 3-axis angular rate sensor 706 and 3-axis accelerometer 708 may output or otherwise report recorded values to the controller 702 for processing, for example via connected wiring or other connections. In some embodiments, the head harness 704 is otherwise similarly configured as the head harness/strap 118 as depicted and described with respect to FIG. 1.

Additionally or alternatively, in some embodiments, the controller 702 is communicatively coupled (e.g., connected via a wire, for example) with a linear actuator 710 and a load cell 712. The linear actuator 710 in some embodiments is configured to generate and/or convert force applied to the head harness 704 to linear movement. In some embodiments, the linear movement is registered via the load cell 712. The load cell 712 is configured to measure a force, for example a resulting from tension caused by the head harness 704. In some embodiments, the load cell 712 is an S-type load cell, for example made of alloy steel and having four strain gauges in a Wheatstone bridge configuration in some embodiments, where a 10-15 volt excitation voltage is supplied to the load cell. In this regard, the load cell 712 deforms when a force (e.g., tension or compression) is applied, thus modifying the voltage output of the strain gauge and yields an output signal. The load cell 712 may be affixed to a point, for example exercise equipment or another stationary mount, to perform the isometric neck strength assessment techniques described herein.

In some embodiments, the load cell 712 is connected to an analog-to-digital converter and a gain amplifier circuit. For example, in some embodiments the load cell 712 is connected to a 24-bit HX711 ADC and an Arduino Nano33 BLE programmable microcontroller formed in a circuit via a load cell amplifier board. The Arduino is powered by a nine volt battery and delivers power to the load cell and amplifier, and also reads the serial output from the amplifier. The microcontroller contains a BLE circuit that is capable of connecting to other BLE devices, for example an external computing device executing an application that utilizes the BLE functionality. In this regard, the microcontroller runs in BLE peripheral mode and accepts connections from BLE central devices, such as an external computing device embodied by a user device. In this regard, the controller 702 may (via the microcontroller, for example) advertise a single BLE service identified by a universally unique ID (e.g., a 128-bit identifier, UUID) that is readable by the central device and connectable to upon selection. The use of BLE functionality may enable lower power consumption and data rates than alterative communications protocols, for example and including traditional Bluetooth.

The controller 702 in some embodiments is a specially configured to control and/or configure one or more computing devices used for the neck strength assessment, receive and/or process data from sensors associated with the neck harness 704 (e.g., the 3-axis angular rate sensor 706 and the 3-axis accelerometer 708) or associated measuring components (e.g., the linear actuator 710 and/or load cell 712). In some embodiments, the controller 702 is a specially configured microcontroller, ASIC, FPGA, processor, and/or the like. The controller 702 may be lightweight and portable such that the entire system 700 is capable of being moved and reconnected to different computing devices. For example, in some embodiments, the controller 702 is specially configured to connect with one or more other devices through a wired and/or wireless connection. The controller 702 may include a universal serial bus (USB) port or other input/output jack that enables software and/or firmware to be uploaded to the controller 702 for subsequent use. In some embodiments, the controller 702 is specially configured to connect wirelessly over one or more radio frequency wireless networks to an associated user device, such as a user's mobile phone. The user may utilize such a user device, for example a mobile device, to select the controller 702 to connect to, send instructions to the controller 702, visualize data received by and/or processed by the controller 702, and/or the like. In some embodiments, the user device is embodied by the computing device 260 and/or 322 with an associated application as depicted and discussed further herein.

In some embodiments, the controller 702 comprises a specially configured Arduino microcontroller with Bluetooth capabilities, for example using Bluetooth Low Energy (BLE) facilitated via a component built into or communicatively coupled to the Arduino microcontroller. The controller 702 in some embodiments is configured to use the Bluetooth module (e.g., a BLE module) to communicate load force measurements to a corresponding application (e.g., on an external computing device, discussed further herein). The application executing on the external computing device may store the data, further transmit the data for storing to a cloud system, and/or provide additional data analysis and/or processing functionality. Such an implementation allows for a low cost and functional neck strength assessment tool.

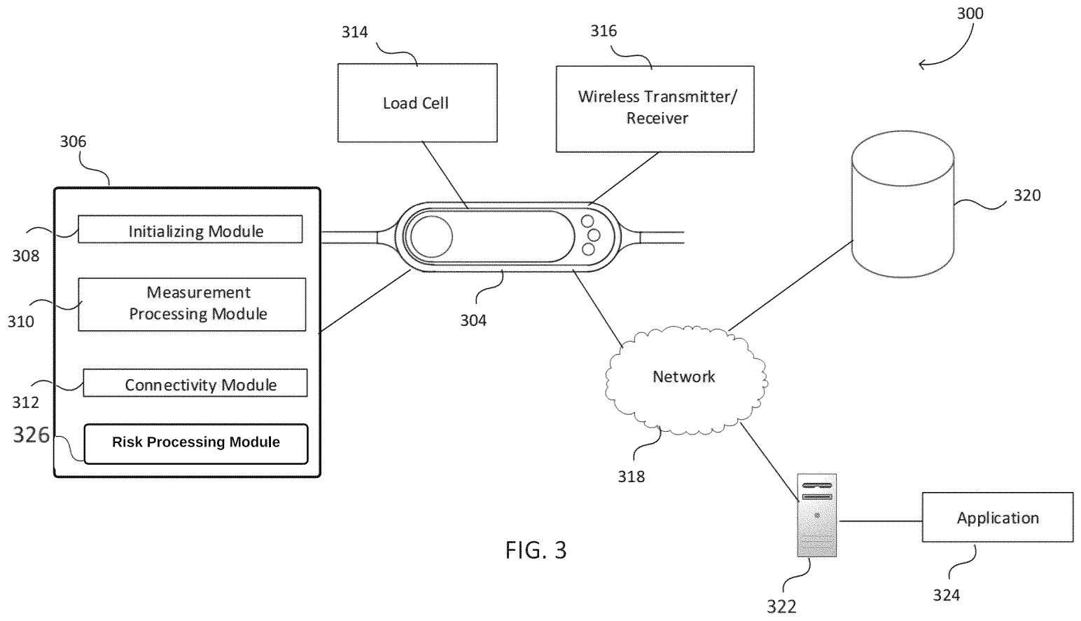

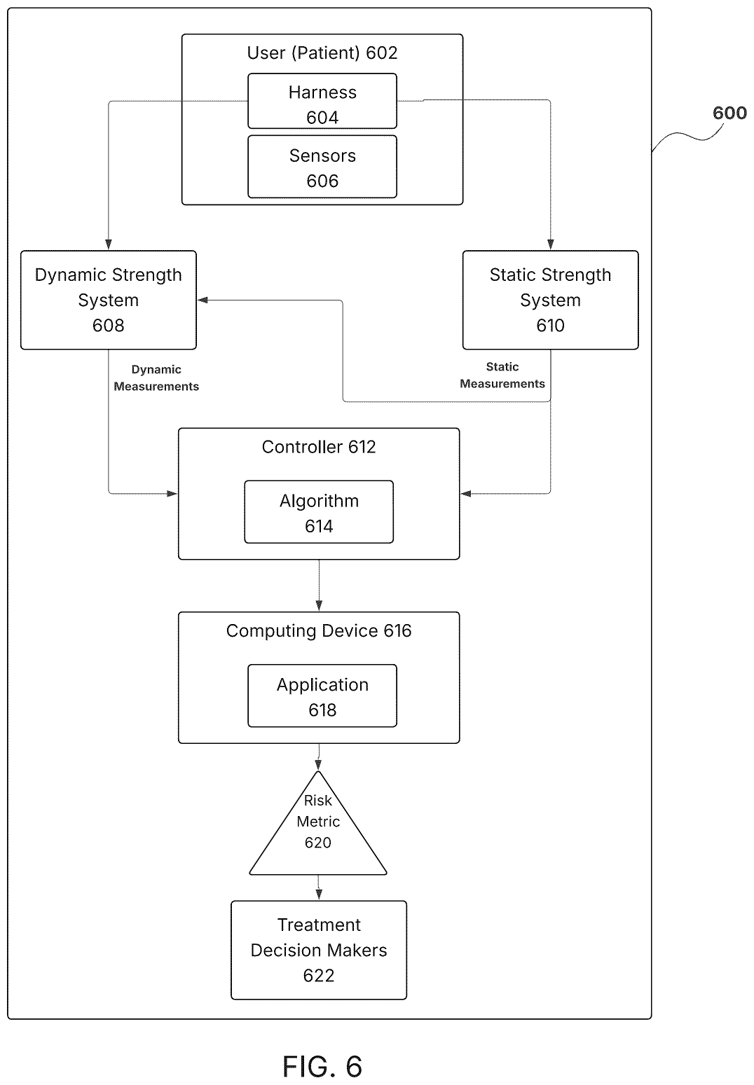

FIG. 6 depicts a block diagram of system components for static and dynamic neck strength measuring in accordance with at least some aspects of the technology described herein. In this regard, the system 600 depicted and described may be an example implementation of the system depicted and described herein with respect to FIG. 1 and/or FIG. 3. As illustrated, the system 600 includes a user 602. The user 602 in some embodiments is a patient, or other individual, that is undergoing treatment or otherwise being tested via the neck strength assessment tool embodied by or otherwise associated with the system 600. The user 602 interacts with a harness 604 and sensors 606. For example, the user 602 may wear the harness 604 to engage the sensors 606, which are configured to measure forces associated with neck strength in one or more directions for example. In some embodiments, the sensors 606 are specially configured to measure a rate of force development, an XYZ direction linear acceleration, and XYZ direction angular acceleration, an XYZ linear velocity, an XYZ angular velocity, an XYZ linear displacement, and/or an XYZ angular displacement.

The system 600 includes a dynamic strength system 608 and a static strength system 610. In some embodiments, the static strength system 610 includes hardware, software, firmware, and/or any combination thereof, that measures static neck strength of the user 602. For example, the static strength system 610 may interface through the harness 604 to engage one or more of the sensors 606. Static strength measurements (“static measurements”) in some embodiments are output to the controller 612 and/or the dynamic strength system 608. In some embodiments, the dynamic strength system 608 includes hardware, software, firmware, and/or any combination thereof, that measures dynamic neck strength of the user 602. The dynamic strength system 608 in some embodiments uses static measurements from the static strength system 610 to set forces used in a dynamic strength measurement, for example forces applied to the user 602 via the harness 604. In some embodiments, the dynamic strength system 608 performs multiple assessments to quantify dynamic measurements including dynamic neck strength and a time to contraction. The dynamic strength system 608 may perform assessments, through the connection with the harness 604 and associated sensors 606, of an anticipated pull and an unanticipated pull. Upon performing an anticipated pull assessment, a user utilizes their neck strength to counteract an expected or timed neck pull coming from the system. Upon performing an unanticipated pull assessment, a user utilizes their neck strength to counteract a random or unexpected neck pull coming from the system. A time-to-contraction for a user may be determined and/or processed for each of these assessments, for example by a specially-configured AI model, ML model, or other algorithm as depicted and described herein. In some embodiments, the static strength system 610 is configured to be connected, via an interface, to the controller, harness 604, and/or the like.

In some embodiments, the static strength system 610 and/or the dynamic strength system 608 are separately connectable (and/or disconnectable from) to a controller 612. In this regard, the dynamic strength system 608 may be connected to the controller 612 before use. The dynamic strength system 608 may similarly be attached to the harness 604 for use.

The measured data (e.g., dynamic measurements and/or static measurements) may be further processed to determine a risk metric. For example, as illustrated, the control 612 receives the dynamic measurements from the dynamic strength system 608, and static measurements from the static strength system 610. The controller 612, via an algorithm 614, may process such data (for example, via an application such as the application 324 depicted and described further herein) together with one or more other data values. For example, in some embodiments, the algorithm 614 processes the dynamic measurements, the static measurements, patient information (e.g., biographic and/or demographic data, and/or the like personal to the user 602), and/or normative data. The algorithm 618 generates and outputs a risk metric, for example the risk metric 620, to the computing device 616. In some embodiments, the risk metric is generated based on static strength data and/or dynamic strength data, or any combination thereof, associated with a user. Additionally or alternatively, in some embodiments, the risk metric is generated based on anticipated assessment data and/or unanticipated assessment data, or any combination thereof, associated with a user.

In some embodiments, the algorithm 618 comprises or embodies an artificial intelligence model, a machine learning model, and/or the like, that generates data based on certain input data. For example, in some embodiments, the algorithm 618 comprises an artificial intelligence model and/or machine learning model that processes data received via interaction with a neck strength assessment device (e.g., the isometric neck strength measurement device including or otherwise associated with the harness 604 and sensors 606). In some embodiments, such data that the artificial intelligence model and/or the machine learning model is specially configured to process as input includes static measurements associated with the user 602, the dynamic measurements associated with the user 602, and/or the like. In some embodiments, such data includes anticipated assessment data and/or unanticipated assessment data. Additionally or alternatively, in some embodiments, the artificial intelligence model and/or the machine learning model is specially configured to process as input includes biographical and/or other data associated with the physical attributes and/or history (e.g., an exercise history, athletic history, injury history, and/or the like) of the user 602. The algorithm 618 (e.g., embodied by the artificial intelligence model and/or machine learning model, for example) may be configured in some embodiments to process such input data and output a corresponding risk metric representing a likelihood of a user (e.g., the user 602) to counteract forces that would result in a head and/or neck injury. In some embodiments, the algorithm 618 (e.g., embodied by the artificial intelligence model and/or machine learning model, for example) may be configured to generate a minimum strength threshold associated with injury risk, where an injury risk metric is determinable based on the minimum strength threshold. For example, a user may be at a higher injury risk in a circumstance where they are further below the threshold.

The risk metric 620 in some embodiments is generated from any of a myriad of available data. For example, in some embodiments, the risk metric 620 is generated by the algorithm 614 based at least in part on data measurements for different measurement types (e.g., a static measurement, an anticipated dynamic measurement, and an unanticipated dynamic measurement) as well as user biographical data. In some embodiments, the risk metric is represented by a number within a range of 0 to 100.

In some embodiments, the computing device 616 executes or otherwise runs an application 618. In some embodiments, the computing device 616 is a mobile device associated with a particular user. The mobile device in some embodiments executes the application 618 embodying a mobile application. For example, in some embodiments, the computing device 616 is a tablet specially configured to execute the application 618. The computing device 616 outputs or otherwise displays the risk metric 620. For example, the risk metric 620 in some embodiments is output to a user of the computing device 616, such as a treatment decision maker 622. The treatment decision maker 622 may utilize the risk metric 620 for any one of a myriad of treatment decisions for an individual, such as the user 602. For example, the treatment decision maker may utilize the risk metric 620 to determine a risk of an injury for the user 602, assign a treatment for the user 602 (e.g., to strengthen the user's neck and reduce injury risk), and/or the like.

In some embodiments, the isometric neck strength measurement device includes a specially configured microcontroller. The isometric neck strength measurement device may be specially configured for any one or more of a myriad of purposes. In some embodiments, for example, the isometric neck strength measurement device is specially configured (e.g., via hardware, software, firmware, and/or any combination thereof) to perform one or more of measuring transducer signals, interpreting user input, coordinating data collection, controlling one or more motors, managing battery power, and/or communicating data to a remote computing device and/or application (e.g., a mobile application or a web application). The isometric neck strength measurement device in some embodiments includes a battery, for example that powers a microcontroller and/or one or more transducers thereof. In some embodiments, the battery is a rechargeable battery, for example charged by via a programming and/or charging port (e.g., a USB-A or USB-C port). The port in some embodiments is on the microcontroller of the isometric neck strength measurement device. Each of the microcontroller, transducer, battery, and/or the like, may be positioned within a housing and/or case (e.g., a plastic case housing) to prevent unwanted contact with such components.

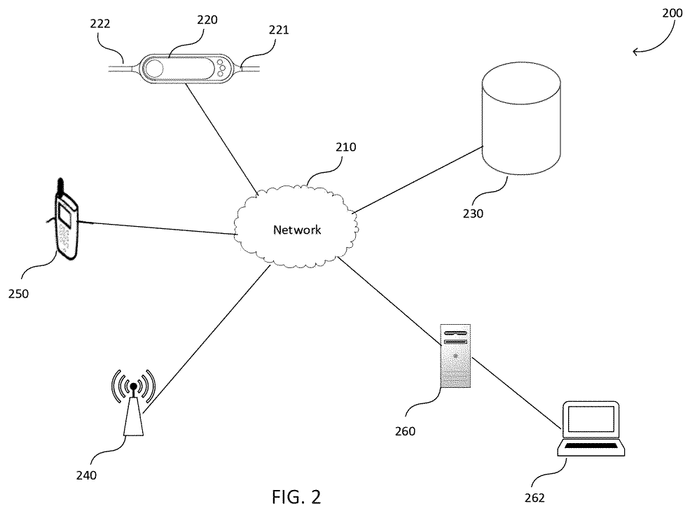

FIG. 2 depicts aspects of an operating environment 200 for an neck strength assessment system in accordance with various embodiments of the present disclosure. Operating environment 200 can include, among other components, a neck assessment device 220, a data repository 230, which can be employed on the isometric neck strength measurement device 220 or remotely, an access point 240, a user device 250, and one or more servers 260. These and other components can be configured to be in operable communication with one another via network(s) 210. The isometric neck strength measurement device 220 can include or be in communication with any computing device, more particularly any portable computing device configured to be implemented in an neck strength assessment system and/or method. The isometric neck strength measurement device 220 can include one or more display components, for example, a display that can present information through visual, auditory, and/or other tactile cues (e.g., a display, a screen, a lamp, a light-emitting diode (LED), a graphical user interface (GUI), and the like. The display component(s) can, for example, present one or more status indicators associated with the isometric neck strength measurement device 220, or present one or more data sets associated with an neck strength assessment system and/or method. In some embodiments, the isometric neck assessment device 220 can have a connection component 221 and/or connection component 222 which can receive and or connect one or more anchor and tensions lines (e.g. 114, 116 of FIG. 1). In some other embodiments, isometric neck strength measurement device 220 can have a first attachment component 221 and a second attachment component 222, where each attachment component is in operable communication with one or more other components and/or integrated with circuitry and/or software of the isometric neck strength measurement device 220, for example a strain gauge, tension scale, or other transducer for measuring neck strength. In some embodiments, the transducer is a tension transducer for measuring neck strength based on corresponding measurements. In some embodiments, the isometric neck strength measurement device includes a tension transducer (e.g., a tension sensor) that further includes at least one ring on either end of a tension transducer. The at least one ring in some embodiments are made of metal, and in some embodiments the at least one ring is made of another material of sufficient rigidity to maintain support to the attached component. In some embodiments, the isometric neck strength measurement device further includes a linear actuator between the transducer (e.g., a tension transducer) and the ring, where the linear actuator is usable to adjust a distance between the transducer and the ring (e.g., to shorten or lengthen the distance). Additionally or alternatively, in some embodiments, the tension transducer is configured to be connectable or otherwise engaged with external equipment (e.g., one or more pieces of exercise equipment) using at least one clip, at least one cable, at least one strap, or other mechanism.

Additionally or alternatively, in some embodiments, the isometric neck strength measurement device further includes an inertia transducer. For example, the isometric neck strength measurement device may include an accelerometer in some embodiments. Additionally or alternatively, in some embodiments, the isometric neck strength measuring device further includes a gyroscope, angular rate sensor, and/or the like. The isometric neck strength measurement device may characterize movements of an individual being measured by the isometric neck strength measurement device. The network 210 can be further connected, via the network, to one or more local or remote servers 260 or computing systems 262, for example portable computing systems such as a tablet or smartphone.

FIG. 3 depicts aspects of a neck strength assessment system 300 used in conjunction with an neck strength measurement device (e.g. 220 of FIG. 2) in accordance with various embodiments of the present technology. The neck strength assessment system 300 can include a plurality of engines, modules, and/or components that can make up a device operation stack 306 of an isometric neck assessment device 304, which can include, but is not limited to: an initialization module 308, a measurement processing module 310, connectivity module 312, and a risk processing module 326. In some embodiments, the device operation stack 306 embodies or includes a controller device that includes hardware, software, firmware, and/or the like to support the operations of the system 300. Among other components not shown, isometric neck strength measurement device 304 (also referred to herein as isometric neck assessment device) can include a user interface and/or user input components for device operation and associated engines and/or modules for their connectivity and operation. As depicted, neck strength assessment system 300 comprises a content repository 320, which can be a plurality of repositories, which can be in operable communication with isometric neck strength measurement device 304 and any associated engines or modules. Content repository 320 can be a local or remote storage device that can contain or host data associated with one or more neck strength assessments and/or with one or more users, for example a user profile.

The initialization module 308 is generally responsible for facilitating communication between one or more sensors (e.g. load cell 314), transmitters (e.g. wireless transmitter/receiver 316), storage, device input/output and their respective subcomponents. The initialization module 308 can initialize (or reinitialize) the isometric neck strength measurement device 304, which in some instances may include calibration and/or recalibration, in response to receiving a signal from an I/O system of the device. For example, a physical input element (such as a button, switch, or the like) can be depressed or a remote control mechanism can be utilized signaling the isometric neck assessment device and connected components should start running. In some embodiments, the isometric neck strength measurement device includes a control panel including such switches, buttons, and/or other physical input element(s).

In some embodiments, the risk processing module 326 is generally responsible for performing a risk assessment of an individual, specifically with respect to a risk of experiencing brain, head, and/or neck injuries. In some embodiments, such a risk assessment includes predicting a minimum threshold of neck muscle force that can counteract or otherwise attenuate forces applied during an impact. In general, anticipatory activation of neck musculature prior to impact has positive effects of decreasing the likelihood of acute head trauma. In other words, a person's engagement of neck muscles in anticipation of impact can be useful in predicting the effects of an impact, as such activation in anticipation reduces the likelihood of traumatic effects. In part, this is due to the activation of the head, neck, and upper back musculature bracing for impact. Thus, improvements in proprioception, neuromuscular control, and muscle fiber contractile speed all affect attenuating force during an unanticipated impact on the body. To promote such reactions, an implementation that dynamically activates the neck musculature while promoting a short latency between contractions.

In some embodiments, the risk processing module 326 configures and/or initializes one or more machine learning algorithms, artificial intelligences, and/or other algorithms that predict a minimum threshold of neck muscle force that counteracts or attenuates forces applied during an impact. In some embodiments, the machine learning algorithm or artificial intelligence comprises a model specially configured to predict the minimum threshold of neck muscle force based on available data associated with the user of the system. In some embodiments, the machine learning algorithm and/or artificial intelligence is embedded in the software application executed by the isometric neck assessment device 304. In some embodiments, the artificial intelligence and/or AI model is embedded as software within the application 324.

The application 324 may be configured to provide various functionality. For example, in some embodiments, the application 324 is configured to provide user sign-up, login, authentication, and/or database storage functionality with respect to an associated cloud database. The user of the mobile application may be an evaluator that is leading a neck strength assessment, or a user performing the neck strength assessment. The application 324 may guide the user through the neck strength assessment protocol, collect the strength readings (e.g., measurements from sensors) from the strength-measurement system, and/or store one or more user's readings to a database (e.g., local or cloud). The application 324 in some embodiments further enables retrieval of stored data values associated with a user.

In an example flow, a user may log in with an email address and password. The user can choose to see a list of recent assessment results or begin a new assessment. When beginning a new assessment, a user may either create a new patient record or search for a previously-entered patient. In either case, the user may then edit a subject's name, height, weight, age, and sex assigned at birth, embodying user or “subject” information. A user may then be taken to a beginning prompt, at which point the application 324 is utilized to connect to a corresponding assessment system (e.g., a controller associated with a network strength assessment tool). Such connection may be needed before proceeding with a neck strength assessment.

Once connected, a user may then select from a set of possible assessment movements to perform from an assessment home screen, for example from a list of cervical extension, cervical flexion, right lateral flexion, or left lateral flexion. The evaluation home page of the application 324 may collect the subject's assessment results thus far. When selecting a movement to assess, corresponding movement instructions will be displayed to read to the subject or for the user to read.

An assessment prompt is displayed, the user may press a button in some embodiments to begin a countdown timer, followed by a subsequent timer (e.g., 5 seconds) during which the subject performs the neck movement and the application 324 streams the measurements (e.g., load cell readings) from the network strength assessment tool (e.g., the isometric neck strength measurement device 304). The application 324 in some embodiments displays the maximum reading measured. In some embodiments, the application 324 repeats for subsequent measurements of the movement, and in some embodiments may average the readings together for all readings or a certain number of readings (e.g., the most recent three readings). It should be appreciated that the user may utilize the application 324 to re-do any of the measurements, review and/or delete individual trial results, and/or otherwise interact with the measurement results.

In some embodiments, the application 324 embodies a mobile application executed on the computing system 322. The computing system 322 comprises a mobile computing device, desktop computing device, and/or the like, that may access the application 324 by running the application 324 on the computing device 322. The application 324 may be a mobile application or a web application, for example. Additionally or alternatively, in some embodiments, the application 324 performs data collection (e.g., from the isometric neck strength measurement device), processing of collected data, and/or outputting of interfaces associated with such data. Such outputting may include displaying one or more user interfaces, the user interfaces including one or more basic summaries and/or interpretations of tests, processes for adding new subjects, selecting existing subjects, manually accessing data entries, downloading of data sets, organizing data, organizing research studies, and/or otherwise controlling access to the software. The application 324 in some embodiments provides access to and/or modification of an individual subject's information, for example a weight, height, age, gender, history of traumatic brain and/or neck injuries, and/or the like. In some embodiments, the computing system 322 embodies a controller that is specially configured to operate one or more functions of the isometric neck strength measurement device 304.

In some embodiments, the application 324 is configured to enable a user to login to a particular account, such that data associated with that account is accessible via the application 324. The logged-in user in some embodiments uses the application 324 to configure the isometric neck assessment device 304 for use. In some embodiments, a user accesses the application 324 to connect to a controller of or associated with the isometric neck assessment device 304, for example by selecting the controller to which to connect from a list of unique IDs corresponding to available controllers found via a wireless search.

In some embodiments, the application 324 is configured to enable registration of a user (e.g., a patient). Additionally or alternatively, in some embodiments the application 324 is configured to enable selection of a registered user for use with the isometric neck assessment device 304. Demographic information, lifestyle information, and/or other information having to do with a user's neck strength and/or potential risk of injury may be stored for subsequent processing. Additionally or alternatively, in some embodiments, the application 324 enables storage of historical data and/or summary information associated with a user, for example a patient's historical neck strength measurements. The summary information may be organized by measurement type (e.g., static measurement data, anticipated dynamic measurement data, unanticipated dynamic measurement data, and/or the like), groupings based on parts of the body utilized (e.g., cervical extension, cervical flexion, left lateral flexion, right lateral flexion, capital extension, capital flexion, cervical rotation, and the like), or groupings based on a pull direction.

In some embodiments, the application 324 is configured to enable a user to select particular measurements to take. For example, a user may interact with the application 324 to initiate measuring of particular measurements (e.g., neck strength in particular directions). In some embodiments, the application 324 initiates a controller to begin taking such measurements in response to a selection by the application 324.

In some embodiments, the application 324 assists a user (e.g., an individual or supervisor) to utilize the isometric neck strength measurement device, for example to perform an isometric and dynamic strength assessment. For example, the application 324 in some embodiments outputs one or more user interfaces with instructions for setting up and/or using an isometric neck strength measurement device to perform an isometric and dynamic strength assessment. Additionally or alternatively, in some embodiments, the application 324 outputs one or more user interfaces with instructions for performing setup of auxiliary equipment (e.g., a wearable head harness, supports structures for connecting the isometric neck strength measurement device or an associated transducer (e.g., a tension transducer) to external equipment, and/or the like). Additionally or alternatively, in some embodiments, the application 324 provides functionality for calibrating an isometric neck strength measurement device, or a tension transducer thereof, for subsequent use. In some embodiments, the application 324 provides a user interface that includes instructions for performing the calibration accordingly. In some embodiments, the isometric neck strength measurement device is calibrated using a set of standardized weights, and/or configuration of one or more force actuators of or associated with the isometric neck strength measurement device to perform isometric neck strength protocols.

In some embodiments, the application 324 leverages artificial intelligence (AI) and/or machine learning (ML) algorithms that enhance functionality thereof. For example, in some embodiments, such integrations allow a user to capture critical neuromuscular data, particularly related to static and dynamic force production and attenuation. This data may be utilized for monitoring health of the head and/or neck complex, particularly in response to bodily impact or whiplash-induced injuries. By analyzing such data, the application 324 can identify individuals at high risk for head and/or neck injuries (e.g., trauma). Additionally or alternatively, preventative measures and/or enhancing the resilience and/or physical readiness for such individuals may be performed. Put differently, for example the application 324 may assess anthropometric measurements to detect the potential risk of TBI or other head and/or neck injuries, and/or to provide an individual with a “no”/“no-go” indication of how to proceed. Thus, embodiments of the present disclosure enable preventative measures to be taken and contribute to the enhancement of resilience and physical readiness, ultimately improving overall safety.

In some embodiments, the application 324 performs a determination of one or more weights (or resistances) to use for a neck strength assessment. The application 324 in some embodiments includes an algorithm, machine learning model, AI model, and/or the like that is specially configured to determine an appropriate weight to use for a particular subject and a particular dynamic neck strength assessment, for example as part of an neck strength assessment. Additionally or alternatively, in some embodiments, the AI model, ML model, or algorithm is specially configured to determine if a minimum threshold of neck muscle force generated can counteract or otherwise attenuate forces applied during an impact. The algorithm, machine learning model, AI model, and/or the like may be configured to determine the appropriate weight to use based on any available data associated with an individual, their history of neck and/or brain injuries, and/or the like. In some embodiments, the AI model, ML model, and/or algorithm is specially configured to process static measurement data and/or dynamic measurement data associated with the user. For example, in some embodiments, static (or isometric) neck strength values and dynamic neck strength values are processed by an AI model, ML model, and/or other algorithm that is specially trained to predict a potential injury risk metric based on such values.

The use of the isometric risk assessment discussed herein together with the minimum threshold of neck muscle force may assist in identifying and/or reducing head and/or neck trauma risks amongst high-risk population groups. While neck-strengthening protocols may be effective and easy to implement, first a reliable, widely available tool must be used to perform neck strength assessment and identify such at-risk individuals. The isometric neck assessment discussed herein thus may be used together with the minimum threshold generating machine learning algorithm or AI further allows for such identification. For example, the machine learning model or AI may be utilized to determine, for a particular individual, a minimum threshold of neck muscle force that can counteract or attenuate forces applied during impact, and the isometric neck assessment described herein may be utilized to determine if the individual's neck strength satisfies their determined minimum threshold.

The risk assessment discussed above, for example as implemented by the risk processing module 326, in some embodiments may be used for research and/or other analytical purposes. For example, in some embodiments, a machine learning or AI model may be utilized to determine an individual's injury risk that is then correlated with the individual's measured neck strength (e.g., measured via the isometric and dynamic neck assessment discussed herein). Additionally or alternatively, in some embodiments, a machine learning or AI model may be used to determine a minimum threshold of neck strength that is then correlated with the individual's measured neck strength (e.g., measured via the isometric neck assessment discussed herein). Cohorts or other groupings of individuals may be linked based at least in part on their injury risk, minimum thresholds, neck strength, or other data.

In some embodiments, the risk processing module 326 includes an adaptive algorithm that continually fine-tunes its determination of risk factors associated with brain and/or neck injury. For example, in some embodiments, the risk processing module processes captured measurements and/or other data of neck strength measurements (e.g., via isometric neck strength assessment described herein) to perform the fine tuning. In this regard, as additional data associated with an individual subject is received, such data may be processed by the algorithm, machine learning model, and/or AI model to improve determination of risk factors associated with neck and/or brain injuries.

Measurement processing module 310 is generally responsible for receiving one or more signals from load cell 314 (or some other digital tension/strain measurement device), associated with an isometric neck contraction input generated or otherwise provided by a user. In some embodiments, the load cell 314 is a specially configured S-type load cell. Measurement processing module 310 can run in conjunction with load cell 314 to convert a physical input associated with an isometric neck contraction of a user to a digital signal. Connectivity module 312 can run in conjunction with one or more wired and/or wireless transmission protocols, for instance with wireless transmitter 316 to transmit collected data and/or measurements associated with an isometric neck assessment (e.g. digital signals) to a repository 320 and/or an external user device (e.g. smartphone, tablet, etc.) or computing system 322. In some embodiments, isometric neck assessment measurements output by isometric neck assessment device 304 can be stored or additionally stored in association with a user and/or clinician profile. Further, one or more external user devices and/or computing systems can have an application running thereon which can act to control and/or operate neck strength assessment system 300 or isometric neck assessment device 304. For example, in some embodiments, the neck strength assessment system 300 and/or isometric neck assessment device 304 is controlled by an external control device. In other embodiments, the neck strength assessment system 300 includes the control device, or the isometric neck assessment device 304 includes the control device. The control device may be specially configured utilizing an application executed thereon.

The system as depicted and described provides a portable, reliable, simple, and rapid neck strength assessment tool, particularly that determines the risk that a patient is at for a TBI based on their static and dynamic strength measurements. The system leverages accurate and repeatable measurement of an individual's (e.g., a patient's, subject's) static and dynamic neck strength. The system further leverages meaningful and actionable calculation of a metric used to determine a patient's risk of suffering a neck and/or head injury (e.g., a TBI), as discussed further herein.

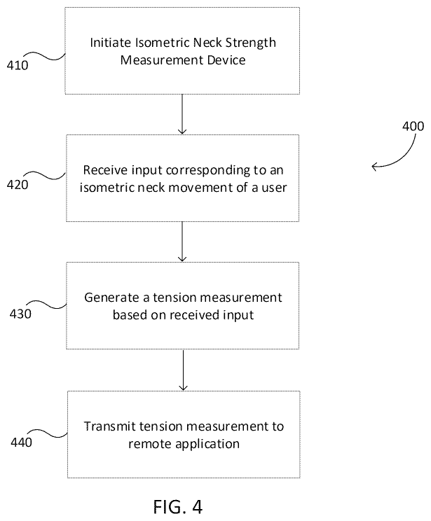

Having described various aspects of neck assessment system(s) and/or device(s), example methods are described below for implementing the forgoing and measuring neck strength of a user, that can include, for example, a isometric neck strength measurement and a dynamic neck strength measurement. Referring to FIG. 4 in light of FIGS. 1-3, a flow diagram illustrating a method 400 for measuring isometric neck strength of a user is provided, in accordance with some aspects of the present technology. The blocks of method 400 and other methods described herein can be carried out by user action, computing processes, digital conversion processes, or a combination comprising the foregoing. In some instances, blocks of method 400 and other methods described herein comprise a computing process that can be performed using any combination of hardware, firmware, and/or software. For instance, various functions can be carried out by a processor executing instructions stored in memory. The methods can also be embodied as computer-usable instructions stored on computer storage media. The methods can be provided by a standalone application, a service or hosted service (standalone or in combination with another hosted service), or a plug-in to another product, to name a few.

At block 410, a neck assessment device or a neck strength measurement device can be initiated and/or calibrated, for instance by an I/O system associated with a neck assessment device and/or a computing system in communication with a neck assessment/strength measurement device. Once initialized and/or calibrated, a user profile and any corresponding user data (e.g. prior assessments and/or measurements, biometric data) can be loaded into local memory of an neck assessment device or another computing device in communication with the neck assessment device. In some instances, initialization by a user/clinician, or computer-based program, can select a isometric neck movement that will be measured.

At block 420, one or measurements corresponding to the neck strength (isometric and/or dynamic) of a user may be taken and further stored. Isometric and/or dynamic neck strength measurements can be taken by neck strength measurement device (e.g. 108 of FIG. 1), that is, neck strength measurement device can receive an input (e.g. an input tension) corresponding to an isometric neck movement of a user, and/or dynamic neck strength and time to contraction through anticipated and unanticipated dynamic pull. Neck strength can be measured by a neck strength measurement device in the following movements corresponding to a user or subject: cervical flexion, cervical extension, capital flexion, capital extension, left lateral flexion, right lateral flexion, left cervical rotation, and right cervical rotation. In some instances, neck strength (isometric and/or dynamic) can be measured in one or more, or all of the forgoing movements, and in some other instances, neck strength can be measured by a subset of the forgoing movements. For example, in some embodiments, neck strength can be measured using a subset of capital flexion, cervical extension, left cervical lateral flexion, and right cervical lateral flexion. Isometric neck movements can be captured, for example using an neck strength assessment system (e.g. 100 of FIG. 1). Various inputs can be received by neck strength measurement device utilizing various attachment points on a head harness/strap and a patient performing neck movements.

In some examples, capital flexion and cervical extension movements can be performed by a user in a tandem stance, with feet shoulder width apart, and arms crossed over the chest. In some other examples, left and right cervical lateral flexion can be performed with the user's feet shoulder width apart. Additionally, these measurements can be taken while the user is in a seated position or standing position. The head strap and/or headgear if the isometric neck strength assessment system (e.g. 104 of FIG. 1) can be secured tightly to the head and/or chin of the user. Each isometric contraction in a given measurement setup can be held for a determined amount of time and/or for a determined number of repetitions, for example held for three seconds and repeated three times per movement. The isometric neck assessment device can subsequently display a numerical value after each contraction which represents the maximal force the user could produce per contraction (e.g. which in some aspects can be measured in kilograms, or other suitable measurement such as N or Nm). In some other embodiments, the isomeric neck assessment device can perform any number of functions or transformative functions on the received measurement data, for example averaging any number of registered measurements and/or determining a peak measurement.

At block 430, based on received input corresponding to a neck movement of a user, the isometric neck strength measurement device or neck assessment device can generate a tension measurement. In some embodiments, the tension measurement corresponds to a maximum contraction of the user for a given isometric neck movement. In some embodiments one or more measurements can be taken and locally stored, and the tension measurement can be an average or other statistical descriptor. In some instances, the tension measurement is displayed on a GUI of the isometric neck strength measurement device and/or isometric neck assessment device.

At block 440, a transmitting component in operable communication with the neck assessment device (e.g. a Bluetooth, RFID, or NFC component) can relay measurement data (such as one or more tension measurements) to another computing device or data storage, for instance an application running on a computing device. In some instances, measurement data can be stored in association with a specified user and/or clinician profile. Additionally, in some other embodiments, other anthropometric measurements and/or other biometric of a user can be entered, stored, and/or otherwise utilized with gathered neck strength measurement data. In some even further embodiments, measurement data can be stored in association with one or more isometric neck movements that were measured,

In some embodiments, the presently disclosed subject matter provides a portable neck strength assessment system and method implementing a neck strength measurement device, and further generating a health risk assessment for a user. The presently disclosed portable neck strength assessment system provides an assessment tool to measure isometric neck strength, which can incorporate additional software components to assess, record, and process isometric and/or dynamic neck strength data. In some instances, a software application can assess and record: isometric neck strength in two planes of movement (sagittal and frontal) through four movements (cervical flexion & extension, right & left lateral flexion). The user of the device and application can further input the following biometric data: height, weight, and neck circumference. This information can then be combined with the isometric neck strength data assessed to produce a health risk assessment for the potential to sustain a mTBI or concussion from an impact or whiplash injury. The information collected on the application is remotely stored and accessible via the user's profile.

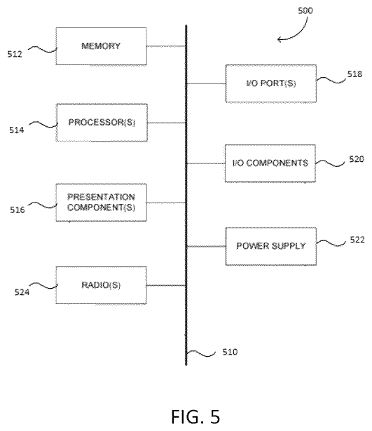

FIG. 5 provides an illustrative operating environment for implementing embodiments of the present technology is shown and designated generally as computing device 500. Computing device 500 is merely one example of a suitable computing environment and is not intended to suggest any limitation as to the scope of use or functionality of the invention. Neither should the computing device 500 be interpreted as having any dependency or requirement relating to any one or combination of components illustrated.

Embodiments of the invention can be described in the general context of computer code or machine-useable instructions, including computer-executable instructions such as program modules, being executed by a computer or other machine (virtual or otherwise), such as a smartphone or other handheld device. Generally, program modules, or engines, including routines, programs, objects, components, data structures etc., refer to code that perform particular tasks or implement particular abstract data types. Embodiments of the invention can be practiced in a variety of system configurations, including hand-held devices, consumer electronics, general-purpose computers, more specialized computing devices, etc. Embodiments of the invention can also be practiced in distributed computing environments where tasks are performed by remote processing devices that are linked through a communications network.

With reference to FIG. 5, computing device 500 includes a bus 510 that directly or indirectly couples the following devices: memory 512, one or more processors 514, one or more presentation components 516, input/output ports 518, input/output components 520, and an illustrative power supply 522. In some embodiments, devices described herein utilize wired and rechargeable batteries and power supplies. Bus 510 represents what can be one or more busses (such as an address bus, data bus or combination thereof). Although the various blocks of FIG. 5 are shown with clearly delineated lines for the sake of clarity, in reality, such delineations are not so clear and these lines can overlap. For example, one can consider a presentation component such as a display device to be an I/O component as well. Also, processors generally have memory in the form of cache. It is recognized that such is the nature of the art, and reiterate that the diagram of FIG. 5 is merely illustrative of an example computing device that can be used in connection with one or more embodiments of the present disclosure. Distinction is not made between such categories as “workstation,” “server,” “laptop,” “hand-held device,” etc., as all are contemplated within the scope of FIG. 5 and reference to “computing device.”

Computing device 500 typically includes a variety of computer-readable media. Computer-readable media can be any available media that can be accessed by computing device 500, and includes both volatile and non-volatile media, removable and non-removable media. By way of example, and not limitation, computer-readable media can comprise computer storage media and communication media.

Computer storage media include volatile and non-volatile, removable and non-removable media implemented in any method or technology for storage of information such as computer-readable instructions, data structures, program modules, or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computing device 500. Computer storage media excludes signals per se.

Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner at to encode information in the signal. By way of example, and not limitation, communication media includes wired media such as a wired network or direct-wired connection, and wireless media such as acoustic, RF, infrared, NFC, Bluetooth and other wireless media. Combinations of any of the above should also be included within the scope of computer-readable media.

Memory 512 includes computer storage media in the form of volatile and/or non-volatile memory. As depicted, memory 512 includes instructions 524, when executed by processor(s) 1014 are configured to cause the computing device to perform any of the operations described herein, in reference to the above discussed figures, or to implement any program modules described herein. The memory can be removable, non-removable, or a combination thereof. Illustrative hardware devices include solid-state memory, hard drives, optical-disc drives, etc. Computing device 500 includes one or more processors that read data from various entities such as memory 512 or I/O components 520. Presentation component(s) 516 present data indications to a user or other device. Illustrative presentation components include a display device, speaker, printing component, vibrating component, etc.

I/O ports 518 allow computing device 500 to be logically coupled to other devices including I/O components 520, some of which can be built in. Illustrative components include a microphone, joystick, game pad, satellite dish, scanner, printer, wireless device, battery, etc.

Many variations can be made to the illustrated embodiment of the present invention without departing from the scope of the present invention. Such modifications are within the scope of the present invention. Embodiments presented herein have been described in relation to particular embodiments which are intended in all respects to be illustrative rather than restrictive. Alternative embodiments and modifications would be readily apparent to one of ordinary skill in the art, but would not depart from the scope of the present invention.

From the foregoing, it will be seen that this invention is one well adapted to attain all ends and objects hereinabove set forth together with the other advantages which are obvious and which are inherent to the structure. It will be understood that certain features and sub combinations are of utility and can be employed without reference to other features and sub combinations. This is contemplated by and is within the scope of the invention.

In the preceding detailed description, reference is made to the accompanying drawings which form a part hereof wherein like numerals designate like parts throughout, and in which is shown, by way of illustration, embodiments that can be practiced. Itis to be understood that other embodiments can be utilized and structural or logical changes can be made without departing from the scope of the present disclosure. Therefore, the preceding detailed description is not to be taken in the limiting sense, and the scope of embodiments is defined by the appended claims and their equivalents.

Various operations have been described as multiple discrete operations, in turn, in a manner that is most helpful in understanding the illustrative embodiments; however, the order of description should not be construed as to imply that these operations are necessarily order dependent. In particular, these operations need not be performed in the order of presentation. Further, descriptions of operations as separate operations should not be construed as requiring that the operations be necessarily performed independently and/or by separate entities. Descriptions of entities and/or modules as separate modules should likewise not be construed as requiring that the modules be separate and/or perform separate operations. In various embodiments, illustrated and/or described operations, entities, data, and/or modules can be merged, broken into further sub-parts, and/or omitted.

The terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth. Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

Unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments ±100%, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Although the foregoing subject matter has been described in some detail by way of illustration and example for purposes of clarity of understanding, it will be understood by those skilled in the art that certain changes and modifications can be practiced within the scope of the appended claims. Many different arrangements of the various components and/or steps depicted and described, as well as those not shown, are possible without departing from the scope of the claims below. Embodiments of the present technology have been described with the intent to be illustrative rather than restrictive. Alternative embodiments will become apparent from reference to this disclosure. Alternative means of implementing the aforementioned can be completed without departing from the scope of the claims below. Certain features and subcombinations are of utility and can be employed without reference to other features and subcombinations and are contemplated within the scope of the claims.

1. A portable neck strength assessment system comprising:
an isometric neck strength device comprising a tension measurement component;
a risk assessment module configured to generate an assessment of an injury risk of an individual using an artificial intelligence model or a machine learning model;
an anchor line having a first end and a second end;
a tension line having a first end and a second end; and
a wearable head harness, wherein the second end of the anchor line is in operable communication with the isometric neck strength device, and wherein the first end of the tension line is in operable communication with the isometric neck strength device, and a second end of the tension line is connected to the wearable head harness.

2. The portable neck strength assessment system of claim 1, wherein the injury risk of the individual is further based at least in part on a neck strength determined using the isometric and dynamic neck strength device.

3. The portable neck strength assessment system of claim 1, wherein the neck strength assessment device comprises a static strength measuring system and a dynamic strength measuring system.

4. The portable neck strength assessment system of claim 1, wherein the artificial intelligence model or the machine learning model generates the assessment of the injury risk based on anticipated assessment data and unanticipated assessment data.

5. The portable neck strength assessment system of claim 1, further comprising an inertia transducer that characterizes movement of the isometric neck strength device.

6. The portable neck strength assessment system of claim 5, wherein the inertia transducer comprises an accelerometer.

7. The portable neck strength assessment system of claim 1, further comprising:
a linear actuator between two rings comprising a first ring at the first end and a second ring at the second end; and
a transducer that adjusts a distance between the two rings.

8. The portable neck strength assessment system of claim 1, wherein the portable isometric and dynamic neck assessment system further comprises:
a microcontroller that measures at least one transducer signal of the isometric and dynamic neck strength device.

9. The portable neck strength assessment system of claim 1, further comprising:
a mobile application executing on a mobile device, wherein the mobile application receives data from the isometric and dynamic neck strength device and uploads the data to a remote storage.

10. The portable neck strength assessment system of claim 1, further comprising:
a web application accessed on a computing device, wherein the web application provides access to uploaded data captured via the isometric and dynamic neck strength device to a database remote from the computing device.

11. The portable neck strength assessment system of claim 1, further comprising:
an application executing on a computing device, wherein the application provides at least one user interface comprising instructions for performing an isometric and dynamic strength assessment using the isometric and dynamic neck strength device, and the wearable head harness.

12. The portable neck strength assessment system of claim 1, further comprising:
an application executing on a computing device, wherein the application provides at least one user interface comprising instructions for calibrating the portable isometric and dynamic neck assessment system for use.

13. The portable neck strength assessment system of claim 12, wherein the portable neck strength assessment system is calibrated using a set of standardized weights.

14. The portable neck strength assessment system of claim 1, further comprising:
an application executing on a computing device, wherein the application comprises an algorithm that uses isometric neck strength data captured using the isometric neck strength device to determine a resistance to use for dynamic neck strength assessment for an individual.

15. The portable neck strength assessment system of claim 1, the risk assessment module comprising:
an application executing on a computing device, wherein the application comprises an algorithm that determines at least one risk factor based at least in part on a neck strength measured for an individual using the isometric and dynamic neck strength device.

16. A method comprising:
measuring a neck strength using an neck strength device that performs an isometric and dynamic neck strength assessment;
generating a minimum threshold using an artificial intelligence model or a machine learning model; and
determining an assessment of an injury risk based on the minimum threshold and the neck strength.

17. The method of claim 16, further comprising:
displaying the assessment of the injury risk via an application executed on a mobile computing device.

18. The method of claim 16, further comprising:
displaying the neck strength via an application executed on a mobile computing device, wherein the isometric and dynamic neck strength device communicates with the mobile computing device to provide the neck strength.

19. The method of claim 16, further comprising:
transmitting, by the isometric and dynamic neck strength device to a mobile computing device, the neck strength; and
uploading, by the mobile computing device using an application executed on the mobile computing device, the neck strength to a database remote from the computing device.

20. The method of claim 16, wherein the artificial intelligence model or the machine learning model is configured to generate the minimum threshold based on anticipated assessment data and unanticipated assessment data
